(19) United States
(12) Patent Application Publication
(10) Pub. No.: US 2018/0055483 A1
(43) Pub. Date: Mar. 1, 2018

(54) Methods and Systems for Ultrasound Controls

(71) Applicant: yoR Labs, Inc., Portland, OR (US)

(72) Inventor: VALERIE HUNTER, PORTLAND, OR (US)

(73) Assignee: yoR Labs, Inc., Portland, OR (US)

(21) Appl. No.: 15/248,881

(22) Filed: Aug. 26, 2016

(51) Int. Cl. A61B 8/00

(52) U.S. Cl. CPC A61B 8/4455; A61B 8/4472; A61B 8/54; A61B 8/467; A61B 8/465

(57) ABSTRACT

An ultrasound probe is provided including a probe body with a sensing face arranged to be held in contact with a patient by a user. The probe provides a plurality of groups of control elements located on two or more exterior surfaces of the probe body sufficiently close to the distal end of the probe such that a user can interact with the control elements while maintaining the scanning position of the ultrasound probe.

### BACKGROUND

An ultrasound image is created by transmitting high frequency sound waves from a transducer in an ultrasound probe into the body and then interpreting the intensity of the reflected echoes in real time. The echos are commonly used to produce two dimensional, three-dimensional, and color flow images of internal anatomical features of patients.

Current ultrasound systems often require an operator to hold an ultrasound probe in one hand while entering information onto a control panel (console) with the other, frequently non-dominant, hand. As the number of functions performed by ultrasound systems increases, the complexity of the user interface, i.e. the console, also increases. This complexity makes it inefficient and time consuming to acquire and optimize images. Additionally, in procedures where the ultrasound is being used as a guide, such as during a needle biopsy, at least three hands are generally used; one to scan, one to hold the needle, and at least one to operate the machine. There is therefore a need for an ultrasound system that provides the operator with a more streamlined method and interface for interacting with, manipulating, and capturing ultrasound images.

### BRIEF SUMMARY

Provided herein are methods and systems for ultrasound, specifically groups of controls on the exterior of an ultrasound housing. In some embodiments, an ultrasound probe housing may include a piezoelectric transducer, a front end analog-to-digital converter, a first processor, and/or a transceiver coupled to the first processor which receives and transmits stored image manipulation signals from user interaction with a group or groups of control elements. In some embodiments, interaction with one or more of the control elements in the group or groups of control elements sends a signal to the first processor to initiate a stored manipulation function associated with a stored control element function. The group or groups of control elements are placed such that the scan position of the ultrasound probe is maintained regardless of the interaction with the control elements.

In some embodiments, a method of manipulating an ultrasound image may include detecting, from a user, an interaction with at least one control element selected from a group or groups of control elements on an exterior surface of an ultrasound probe; correlating the interaction with the at least one control element with a stored image manipulation function; and/or applying the image manipulation function associated with the interaction with the at least one control element selected from the group or groups of control elements to an active ultrasound image.

The group or groups of control elements may be located within 4, 5, 6, 7, 8 cm from the distal end of the ultrasound probe. In some embodiments, the group or groups of control elements begin at least 1, 2, 3, 4, 5, 6, 7, 8, 9, 10 mm from a distal end of the ultrasound probe. Control elements may include buttons, slide bars, scroll wheels, touch pads, switches, finger operated joysticks, or any combination thereof. Such control elements may be manipulated by any means generally used including, but not limited to, a swipe, tap, double tap, a switch, flipped, press and hold, scroll and the like. Manipulation of one or more of the control elements may initiate a discrete action or may activate a menu that may be scrolled through. The control elements may be programmable by the user or may be static with a fixed function. In further embodiments, some of the control elements may be static while other control elements are programmable. In some embodiments, one or more control elements may have the same or different functions. Control elements may be manipulated singly, in a group, or in a particular pattern among a plurality of control elements.

In some embodiments, manipulation with one or more of the control elements in the group or groups of control elements triggers an alert such as an audible signal, a vibration, a light, or a color change. The alert may be the same or different for each type of interaction or manipulation of the control elements.

To the accomplishment of the foregoing and related ends, certain illustrative aspects of the system are described herein in connection with the following description and the attached drawings. The features, functions and advantages that have been discussed can be achieved independently in various embodiments of the present disclosure or may be combined in yet other embodiments, further details of which can be seen with reference to the following description and drawings. This summary is provided to introduce a selection of concepts in a simplified form that are elaborated upon in the Detailed Description. This summary is not intended to identify key features or essential features of any subject matter described herein.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an embodiment of processing raw input received from a phased array with a beamforming apparatus as described herein.

FIG. 2 illustrates an aspect of the conversion of sound waves sent and received by a transducer in an ultrasound probe into a digital image.

FIG. 3 illustrates a top down view of an ultrasound probe with groupings of control elements as described herein.

FIG. 4 illustrates a side view of an embodiment of an ultrasound probe with groupings of control elements as described herein.

FIG. 5 illustrates a perspective view of an ultrasound probe being held by a user.

FIG. 6 illustrates an embodiment of a perspective view of an ultrasound probe with a plurality of control elements.

FIG. 7 illustrates a view from the distal end of an embodiment of an ultrasound probe with a plurality of control elements as described herein.

FIG. 8 illustrates a routine for transforming mechanical interaction with control elements on the exterior of an ultrasound probe into a digital signal in accordance with one embodiment.

FIG. 9 illustrates a system 800 for information input and processing in accordance with one embodiment as described herein.

### DETAILED DESCRIPTION

### Glossary

“Distal end” in this context refers to the end of an ultrasound wand located closest to the part of the body being scanned.

“Front end” in this context refers to a set of analog signal conditioning circuitry that uses sensitive analog amplifiers to provide a configurable and flexible electronics functional block which is used to interface a variety of sensors to an analog to digital converter.

“Front end analog-to-digital converter” in this context refers to a means for converting an analog input signal to a digital signal.

“Proximal end” in this context refers to the end of the ultrasound wand traditionally attached to computerized ultrasound equipment and furthest away from the body part being scanned.

“Slide bar” in this context refers to a manipulable button or other control element attached to the ultrasound wand by a first end which may be used to scroll through a menu on a display and select one or more items.

Described herein is an ultrasound imaging system and means for recording, manipulating, comparing, labeling, and/or documenting information received from an ultrasound probe using a plurality of control elements on the exterior of an ultrasound probe housing. Further provided is a system including an ultrasound probe comprising a transducer configured to transmit an ultrasound signal to a target object, receive an ultrasound echo reflected from the target object, and a first processor configured to form and transmit an ultrasound image corresponding to the target object to a display. Such transmission may be wired or wireless.

During an ultrasound scan, a user scans with one hand while the other, usually non-dominant, hand remains on an ultrasound control panel (console) to operate controls to optimize and manipulate an image. By moving frequently used controls to the ultrasound probe, the need to manipulate a control screen or other user interface decreases. Additionally, in the instance of guided procedures, moving frequently used controls to the ultrasound probe decreases the need for an additional person to manipulate the console. Such control elements located on the ultrasound probe are intended to be easy to manipulate using the fingers of the hand holding the ultrasound probe. In some embodiments, the user is able to easily reach the on-probe controls with the hand holding the probe without having to change the position of the scanning ultrasound probe.

As shown herein, a plurality of control elements including, but not limited to, buttons, scroll wheels, slide bars, touch pads, switches, finger operated joysticks, and the like are located near the distal end of two or more exterior surfaces of the ultrasound housing such as a top side, bottom side, left or right side of the housing, in such a position as to be readily manipulable with the hand holding the ultrasound probe. In some embodiments, the control elements may be within reach of fingers not supporting the probe such that manipulation of the controls minimally effects the scan of the probe. In additional embodiments, the control elements may be clustered together such that the cluster or grouping of control elements on a single surface of an ultrasound probe housing is no more than 5, 4, 3, 2 cm or a fraction thereof in width. In some embodiments, the plurality of control elements may be located in two or more clusters or groupings, one near the distal end of a top side and the other near the distal end of the right or left side, wherein the top side and the right or left side are orthogonal to each other. In additional embodiments the location of a cluster of control elements may begin at least 3, 4, 5, 6, 7, 8, 9 mm from the distal end of the ultrasound probe or any fraction thereof. In further embodiments, the location of a cluster of control elements may begin no more than 5 mm from the distal end of the ultrasound probe. In additional embodiments, the groups of control elements may be located in their entirety within 5 cm of the distal end of the ultrasound probe. In some embodiments, the beginning edge of the first control element on a side of the ultrasound probe may begin no more than 5, 4, 3, 2, 1, mm from the edge of the probe, as measured from a right side edge of the ultrasound probe wherein the right side edge is the orthogonal edge between the top and right sides of the ultrasound probe.

Control elements may comprise any suitable technology such as, but not limited to, electromechanical switches, membrane switches, inductive or capacitive contact switches, and the like. Control elements may be activated alone or in combination with the same or other groups to achieve the desired manipulation. In some embodiments, groups of two or more including 3, 4, or 5 control elements may be placed on one or more sides of the exterior of the ultrasound probe. Such groups of control elements may have the same or different functions. Once the control element(s) are activated, the processor performs the device function associated with the stored image manipulation or control element function. The control elements may be used alone or in one or more combinations to input the desired commands. Such commands include, but are not limited to, freeze/unfreeze, save, measure, print, select, insert text cursor, advance to next, text, display list of operations, display list of tools and/or activate pre-assigned operation. Pre-assigned operations may include, but are not limited to operations useful for a Doppler exam such as spectral, color, power m-mode and continuous wave (cw). Other pre-assigned operations include, but are not limited to: cine, harmonics, and compound imaging. In some embodiments, controls on an ultrasound probe may access a list of tools useful in an ultrasound exam including, but not limited to, frame rate, adjust field of view, adjust time gain compensation, adjust dynamic range, adjust low pass filter, adjust Doppler gain, and adjust master gain. In additional embodiments, control elements may activate one or more image optimization functions such as, but not limited to, a focal zone, a time gain compensation, a dynamic range, a frame rate, a Doppler gain, and a field width of the active ultrasound scan. Such operations may be individually assigned to a single control element or combination of control elements, or may be used for selecting elements from a larger menu that may be scrolled through.

In some embodiments, one or more of the controls such as one or more of the slide bars or other sliding controls may be used to scroll through and select items from one or more menus on the user interface. Exemplary information that may be selected on the user interface includes, but is not limited to, the direction of the scan; whether the lesion is a cyst, mass, duct or blood vessel; whether it is located anterior to or posterior to an anatomical landmark; its size and the like. In some embodiments, sliding controls may be used to select from commonly used labels for a specific anatomical feature. For example, a word bank for a breast study may include, but is not limited to, RAD, ARAD, SAG, TRAN, Axilla, Axillary tail, Subareolar, Inframammary fold, Anterior Axillary line, Middle Axillary line, Posterior Axillary line, Palp, Palp area, Tract, Calcifications, Heel rock, Toe rock, Skin fascia, Chest wall, Lateral to, Medial to, Cyst, Foam cyst, FA, Mass, Halo, Typical, swirling echoes, and vessel. A word bank for a thyroid study may include, but is not limited to, Right, Left, SAG, TR, Isthmus, upper pole, mid gland, lower pole, Esophagus, Cyst, Cystic changes, Microcalcifications, Thyroid bed, and Lower pole obscured by clavicle. In another example, a word bank for a cervical node study may include, but is not limited to, Cyst, Cystic changes, Calcification, Microcalcifications, Hilar flow, Cortical flow, and Thyroid bed.

In additional embodiments, one or more of a plurality of groups of control elements on the ultrasound probe may be used to accept a structured label. Structured labels may be used where a scan generally proceeds in a standard order and typical images are acquired. Standardized labels may appear in order and the user merely accepts the labels. Common scan types for structured labeling would include, but are not limited to, obstetrics, abdomen, carotid, lower extremity venous among others. The order and the labels in the structured label list may be fixed or customizable. In some embodiments, structured labels for an obstetric scan for maternal and fetal anatomy may be customized to be presented in the order the sonographer usually scans: for example, cervix, placenta trans, placenta long, ventricles, CSP, CM, orbits, face, N/L and profile, among others. For a thyroid study, the structured labels presented may include, but are not limited to, Right, RUP TR, R mid TR, RLP TR, R SAG lat, R SAG ML, R SAG med, R Level IV LN, Isthmus TR, Isthmus SAG, Left, LUP TR, L mid TR, LLP TR, L SAG med, L SAG ML, L SAG lat, and L Level IV LN. The structured labels for a cervical node study may include, but are not limited to, Right Neck with sub labels including Level I, TR; Level I, LG; Level IIA, TR; Level IIA, LG; Level IIB, TR; Level IIB, LG; Level III, TR; Level III, LG; Level IV, TR; Level IV, LG; Level V, TR; Level V, LG; Level VA, TR; Level VA, LG; Level VI TR; Level VI, LG; Thyroid bed, TR; and Thyroid bed, LG. The Left Neck may appear with sub labels including, but not limited to, Level I, TR; Level I, LG; Level IIA, TR; Level IIA, LG; Level IIB, TR; Level IIB, LG; Level III, TR; Level III, LG; Level IV, TR; Level IV, LG; Level V, TR; Level V, LG; Level VA, TR; Level VA, LG; Level VI TR; Level VI, LG; Thyroid bed, TR; and Thyroid bed, LG. Labels may also include numbers indicating the presence of a plurality of lesions and providing reference tags for follow up studies of each lesion. In some embodiments, each lesion may be numbered automatically.

In further embodiments when one or more control elements are activated an alert or signal is transmitted to the user indicating that the command has been received. Such signals may be transmitted by the ultrasound probe and/or other parts of the ultrasound system such as the console and/or image display. Such a signal may be one or more of a single or series of audible signals, vibrations, lights, color change, and the like. In some embodiments, different signals may be activated for different actions. In other embodiments, the same signal may be activated, regardless of the action taken.

The control elements may be any shape desired. In some embodiments, they may all be the same shape. In other combinations they may be a plurality of shapes which may move in the same or different ways. The control elements may protrude and/or be level with the ultrasound probe. In some embodiments, the control elements may be covered by a membrane or other protective seal to prevent contamination.

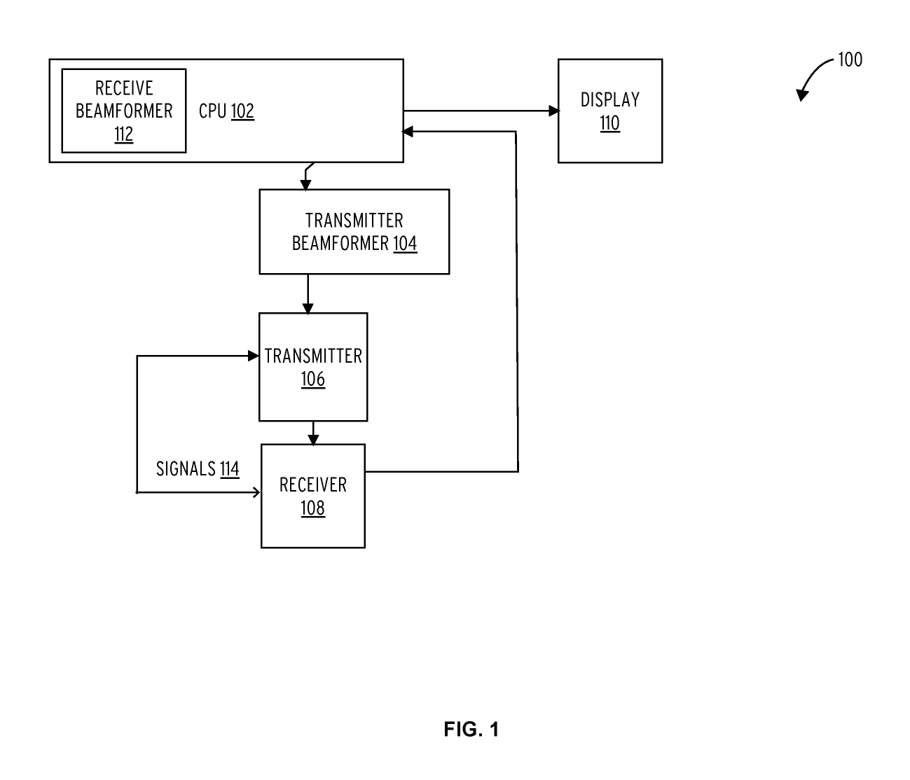

Turning to the figures, as shown in the phased array with beamforming apparatus in FIG. 1, a CPU 102 sends instructions to a transmitter beamformer 104 that a signal should be sent from the transmitter 106. The transmitter 106 emits or instructs a transducer (not shown) to emit a series of pulsed signals 114. The series of pulsed signals 114 are captured by a receiver 108 which may be the same or a different transducer than the transmitter 106. In some embodiments the transmitter 106 and the receiver 108 are the transducers or sensors. They may be the same or different transducers located in a phased array or in a probe. The echo received by the receiver 108 is then sent to the CPU 102 for receive beamformer 112. The CPU 102 may process the received data in real-time during a scanning/signal capture event or after a delay. Processed frames of data are presented on a display 110. In some embodiments, the CPU 102, transmitter beamformer 104, transmitter 106, and receiver 108, may all be contained in an ultrasound probe. In other embodiments, they may be in one or more different wireless and/or wired devices.

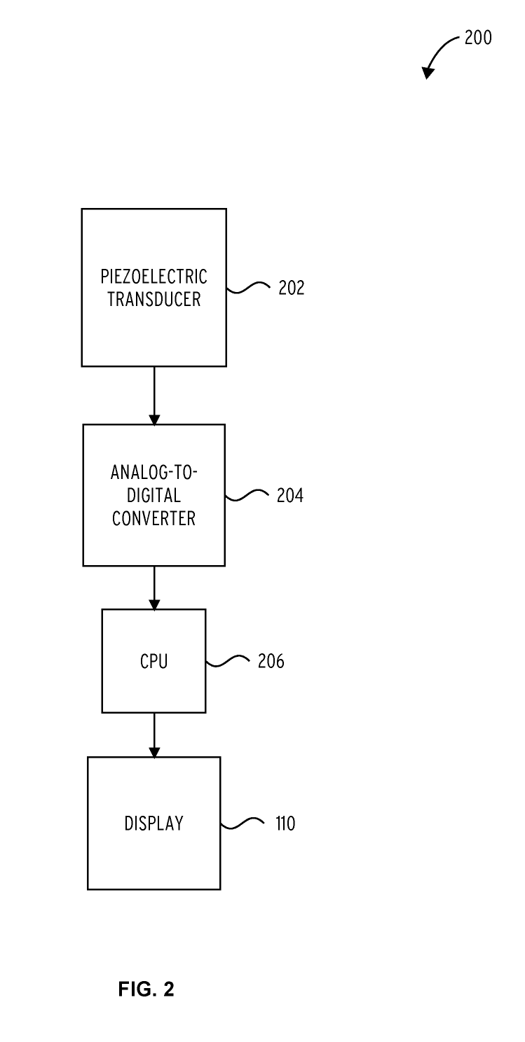

Referring to FIG. 2, a piezoelectric transducer 202 in an ultrasound probe converts an electric signal to rapid mechanical vibrations and transmits the mechanical vibrations through the ultrasound probe into the body. Return vibrations are captured by the piezoelectric transducer and converted by the front end analog-to-digital converter 204 to a digital image. The converted signals are then processed by the CPU 206 and the resulting image is communicated to a display 110. The ultrasound probe may be wired or wireless. In some embodiments, the ultrasound probe may further comprise a battery.

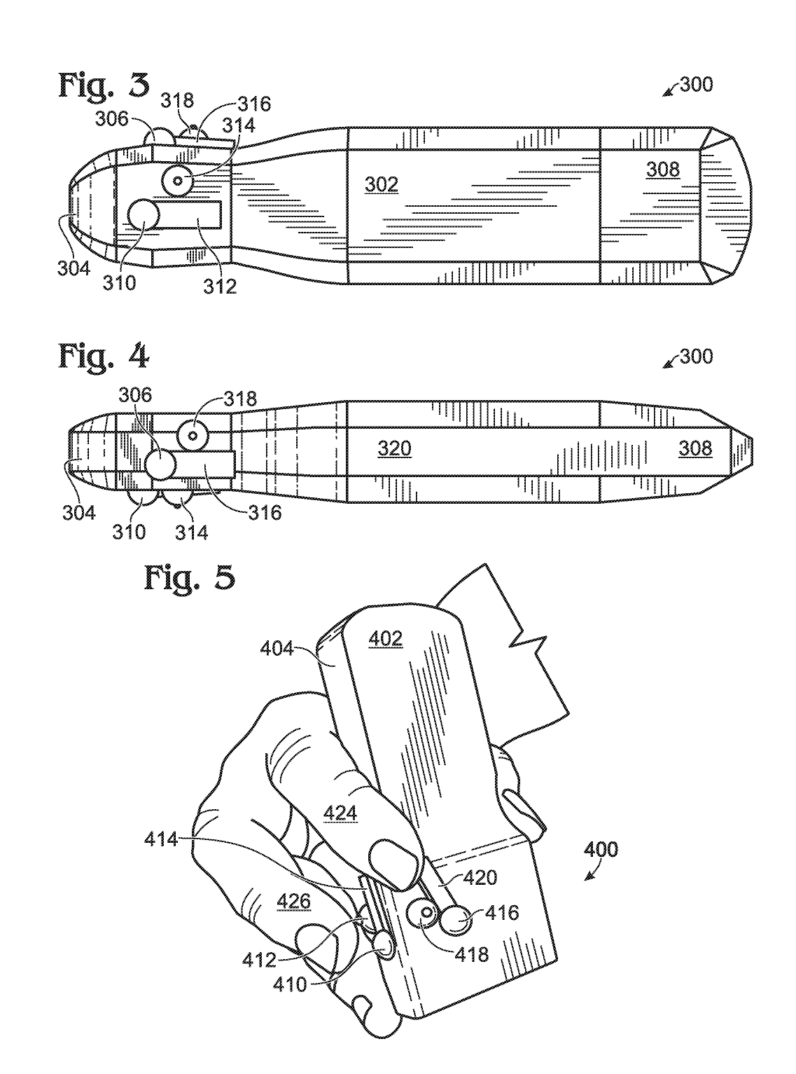

Referring to FIG. 3 which illustrates a top down view and FIG. 4 which illustrates a right side view of an ultrasound probe, a top side 302 comprises a distal end 304 and a proximal end 308, wherein the distal end 304 is designed to be placed upon the skin of a patient or object being scanned. A plurality of control elements which may be manipulated using the fingers holding the ultrasound probe are located near the distal end 304, allowing for one handed manipulation of the probe and controls. Such control elements 306, 310, 312, 314, 316 and 318 may be located on a top side, bottom side, right side and/or left side or any combination thereof as oriented by the top side 302. Such control elements may be used alone or in combination to interact with an ultrasound image taken by the ultrasound probe 300. In some embodiments, each control element may respond to a plurality of interactions including, but not limited to, a switch, flipped, tap, double tap, swipe, press and hold, scroll and the like. In additional embodiments, interaction with two, three, four or more control elements in the same or different groupings concurrently or in combination may initiate additional commands or interactions with the ultrasound image. In some embodiments, some or all of the control elements may be programmable. For example, the most common controls may be fixed and additional controls may be programmable. In other embodiments, the control elements are fixed. The clusters of the control elements on the top side, bottom side, right side or left side may have the same or different functionalities. In some embodiments, some or all of the control element functions may be set by the user. In other embodiments, some or all of the control element functions are predetermined.

In some embodiments, a control element may be a slide bar or scroll as shown at fifth control element 312 and sixth control element 316. Such control elements may allow the user to choose between a plurality of possible states. In some embodiments, manipulation of a slide bar or scroll may allow a user to scroll through a range of possible states, word bank, structured labels, or other menus on a control interface. Such a menu may include, but is not limited to, paging through a cine loop, enlarging/reducing a selected image, adjusting time gain compensation, adjusting volume control, scrolling through a list of possible commands, scrolling through a list of labels in a word bank, scrolling through images in a stack of thumbnails, or adjusting any console function. In further embodiments, additional menus manipulable by the control elements on the probe may include the most used controls for a specific type of scan. The controls listed in the drop down menus may vary depending on the type of scan selected or may be the same for all scans. In some embodiments, a drop down menu may include user preferences which may be saved under a user id and automatically populate when a user logs in. While such user preferences may refer to any desired action, in some embodiments, the user preferences may include the number of frames in a cine loop, dynamic range, field width or frame rate present for particular exam types.

FIG. 5 is a perspective view of an embodiment of an ultrasound probe 400 being held by a user. Although a right hand is depicted, the probe may be used by either a right or left handed user. In some embodiments, the control elements may be within reach of fingers not supporting the probe such that manipulation of the controls minimally affects the scan of the probe.

The control elements 410, 412, 414, 416, 418 and 420 are readily accessible using the fingers such as 424 and 426 on the hand using the probe. In some embodiments, such control elements may be located within about 4, 5, 6, 7, or 8 cm from the distal end 406 of the ultrasound probe 400. In additional embodiments, the control elements may be located in their entirety between about 0.5 to about 5 cm from the distal end of the ultrasound probe. The groupings of a plurality of control elements, such as the group of control elements located on the top of the ultrasound probe or those located on the right side of the ultrasound probe may be about 0.5, about 1, 2, 3, 4 cm wide so that all of the control elements in a particular grouping are within reach of the user's finger as shown at 424 or 426 respectively. In some embodiments, the grouping of control elements may be no longer than 0.5, 1, 2, 3, 4, 5 cm long such that all control elements within a single grouping of control elements such as the grouping of 416, 418 and 420 on the top side or the grouping of 410, 412 or 414 on the right side are within the specified distance.

In some embodiments, the plurality of control elements may be located in two or more clusters, one on the distal end of a top side 402 and the other on a distal end of a right side 404 wherein the top side 402 and the right side 404 are orthogonal to each other and the placement of the cluster of control elements is within about 2, 3, 4, 5 mm or a fraction thereof from the right side edge 422 shared by the top side 402 and the right side 404. In additional embodiments the location of a cluster of control elements may begin at least 3, 4, 5, 6, 7, 8, 9 mm from the distal end of the ultrasound probe or any fraction thereof.

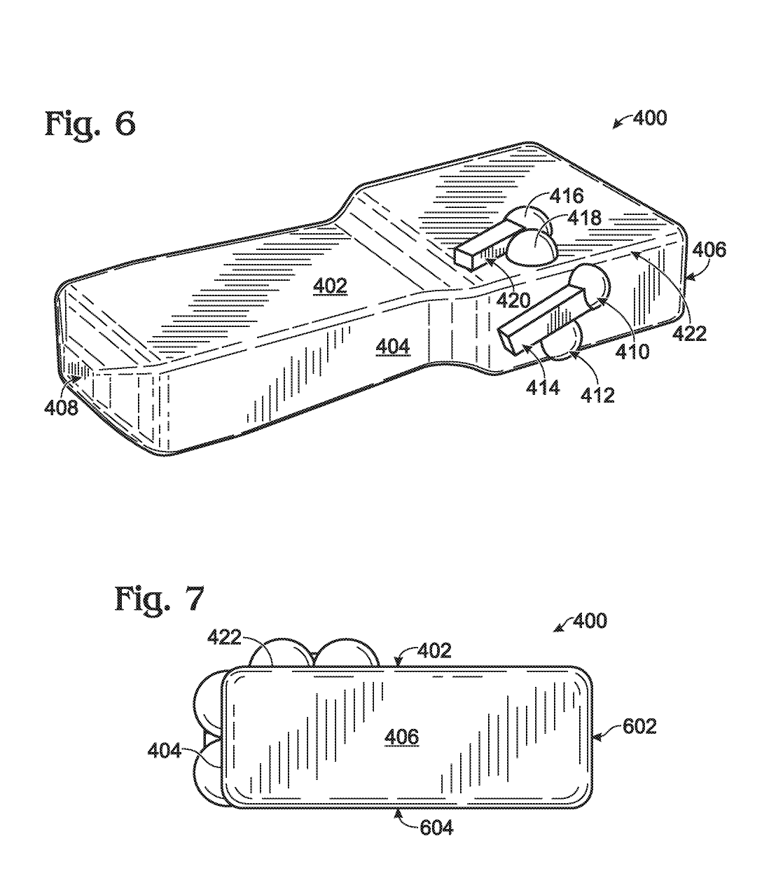

Referencing the illustrations depicted in FIG. 6 and FIG. 7, an exemplary ultrasound probe 400 comprises a top side 402, a bottom side 604, a right side 404 and a left side 602 with a distal end 406 and a proximal end 408. As shown in FIG. 5, the ultrasound probe 400 comprises a plurality of groups of control elements. A first group of control elements on a right side 404 of the ultrasound probe 400 comprises a first control element 410, a second control element 412, and a third control element 414. A second group of control elements on the top side 402 comprises a fourth control element 416, a fifth control element 418, and a sixth control element 420. While the control elements are referenced as a first, second, third, fourth, fifth and sixth control element respectively, there may be more or fewer control elements in additional groups distributed near the distal end 406 of the ultrasound probe 400. Interaction with the control elements may occur in any order or pattern depending on the desired stored image manipulation function associated with a particular interaction with one or more control elements.

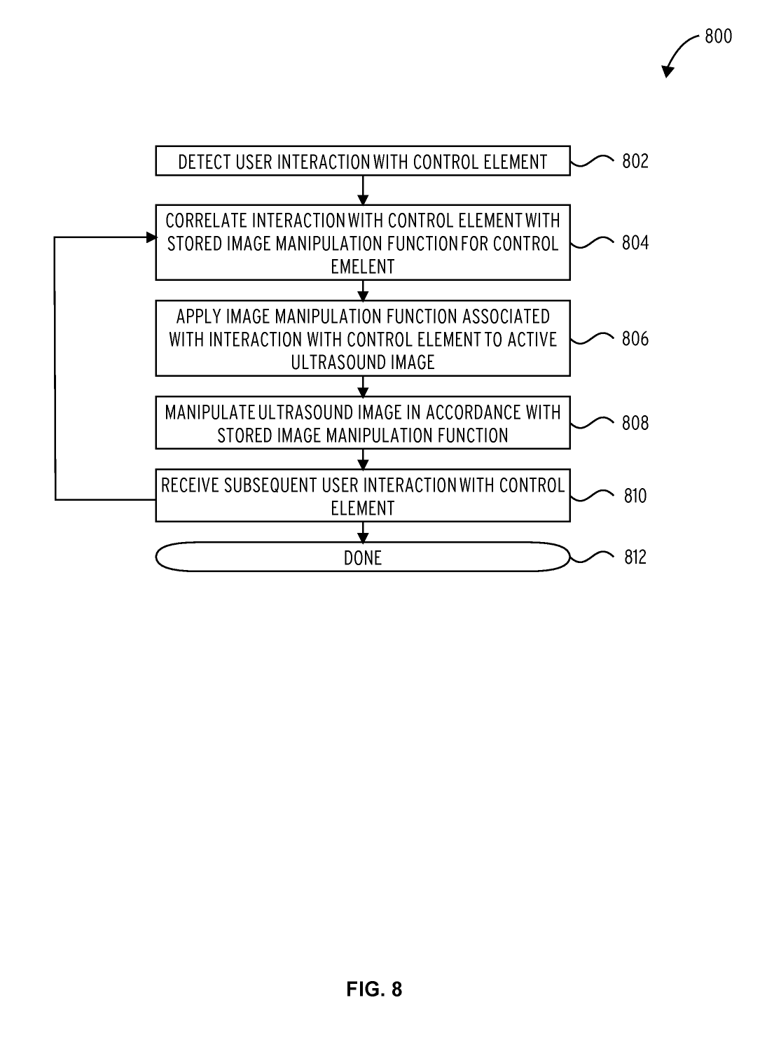

Referring to FIG. 8, in block 802, routine 800 detects a user interaction with a first control element. In block 804, routine 800 correlates the interaction with a first control element with a stored image manipulation function. In block 806, routine 800 applies the image manipulation function associated with the interaction with the at least one control element to an active ultrasound image. In block 808, routine 800 manipulates the ultrasound image in accordance with the stored image manipulation function. In block 810, routine 800 detects a subsequent user interaction with a second control element and the routine 800 repeats until no further user interactions with control elements are detected. In some embodiments, the first control element and/or the second or additional control element may be a plurality of control elements or a pattern of interactions with control elements.

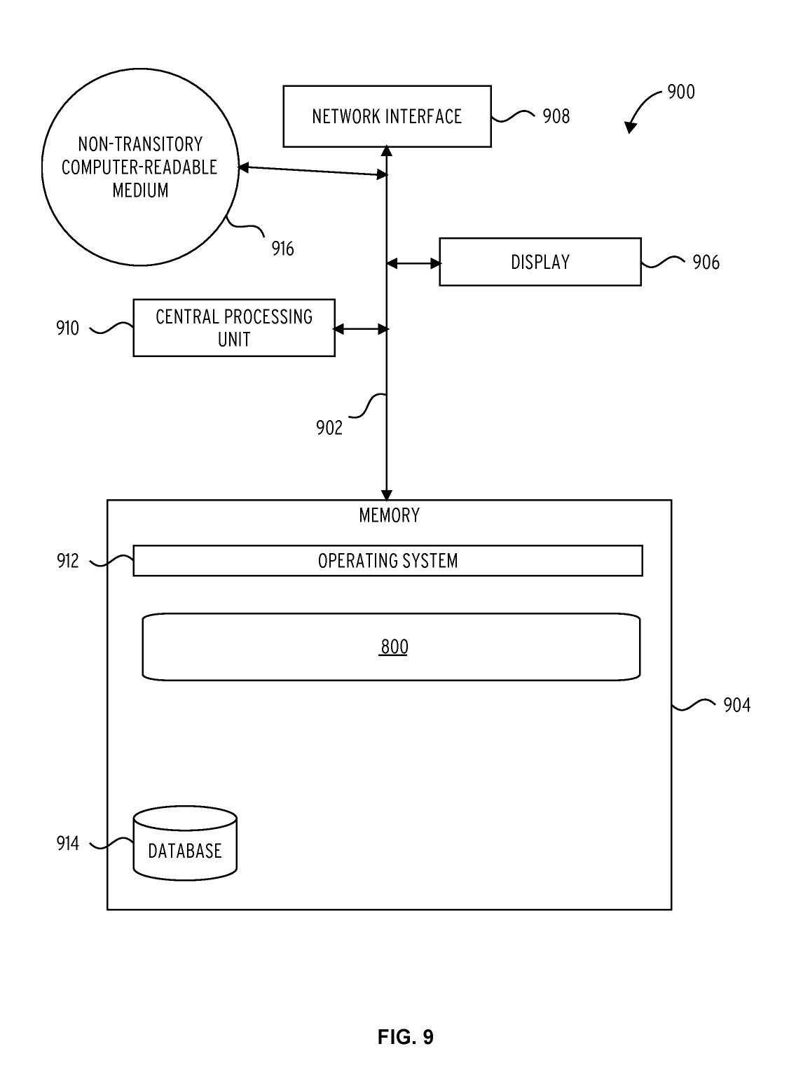

FIG. 9 illustrates several components of an exemplary system 900 in accordance with one embodiment. In various embodiments, system 900 may include a desktop PC, server, workstation, mobile phone, laptop, tablet, set-top box, appliance, or other computing device that is capable of performing operations such as those described herein. In some embodiments, system 900 may include many more components than those shown in FIG. 9. However, it is not necessary that all of these generally conventional components be shown in order to disclose an illustrative embodiment. Collectively, the various tangible components or a subset of the tangible components may be referred to herein as “logic” configured or adapted in a particular way, for example as logic configured or adapted with particular software or firmware.

In various embodiments, system 900 may comprise one or more physical and/or logical devices that collectively provide the functionalities described herein. In some embodiments, routine 800 may comprise one or more replicated and/or distributed physical or logical devices.

In some embodiments, system 900 may comprise one or more computing resources provisioned from a “cloud computing” provider, for example, Amazon Elastic Compute Cloud (“Amazon EC2”), provided by Amazon.com, Inc. of Seattle, Wash.; Sun Cloud Compute Utility, provided by Sun Microsystems, Inc. of Santa Clara, Calif.; Windows Azure, provided by Microsoft Corporation of Redmond, Wash., and the like.

System 900 includes a bus 902 interconnecting several components including a network interface 908, a display 906, a central processing unit 910, and a memory 904.

Memory 904 generally comprises a random access memory (“RAM”) and permanent non-transitory mass storage device, such as a hard disk drive or solid-state drive. Memory 904 stores an operating system 912 and at least one routine 700.

These and other software components may be loaded into memory 904 of system 900 using a drive mechanism (not shown) associated with a non-transitory computer-readable medium 916, such as a floppy disc, tape, DVD/CD-ROM drive, memory card, or the like.

Memory 904 also includes database 914. In some embodiments, system 900 may communicate with database 914 via network interface 908, a storage area network (“SAN”), a high-speed serial bus, and/or via the other suitable communication technology.

In some embodiments, database 914 may comprise one or more storage resources provisioned from a “cloud storage” provider, for example, Amazon Simple Storage Service (“Amazon S3”), provided by Amazon.com, Inc. of Seattle, Wash., Google Cloud Storage, provided by Google, Inc. of Mountain View, Calif., and the like.

References to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple one. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other.

“Logic” refers to machine memory circuits, non-transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter). Those skilled in the art will appreciate that logic may be distributed throughout one or more devices, and/or may be comprised of combinations of memory, media, processing circuits and controllers, other circuits, and so on. Therefore, in the interest of clarity and correctness logic may not always be distinctly illustrated in drawings of devices and systems, although it is inherently present therein.

The techniques and procedures described herein may be implemented via logic distributed in one or more computing devices. The particular distribution and choice of logic will vary according to implementation. Those having skill in the art will appreciate that there are various logic implementations by which processes and/or systems described herein can be effected (e.g., hardware, software, and/or firmware), and that the preferred vehicle will vary with the context in which the processes are deployed. “Software” refers to logic that may be readily readapted to different purposes (e.g. read/write volatile or nonvolatile memory or media). “Firmware” refers to logic embodied as read-only memories and/or media. Hardware refers to logic embodied as analog and/or digital circuits. If an implementer determines that speed and accuracy are paramount, the implementer may opt for a hardware and/or firmware vehicle; alternatively, if flexibility is paramount, the implementer may opt for a solely software implementation; or, yet again alternatively, the implementer may opt for some combination of hardware, software, and/or firmware. Hence, there are several possible vehicles by which the processes described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations may involve optically-oriented hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood as notorious by those within the art that each function and/or operation within such block diagrams, flowcharts, or examples can be implemented, individually and/or collectively, by a wide range of hardware, software, firmware, or virtually any combination thereof. Several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, flash drives, SD cards, solid state fixed or removable storage, and computer memory. In a general sense, those skilled in the art will recognize that the various aspects described herein which can be implemented, individually and/or collectively, by a wide range of hardware, software, firmware, or any combination thereof can be viewed as being composed of various types of “circuitry.” Consequently, as used herein “circuitry” includes, but is not limited to, electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), circuitry forming a memory device (e.g., forms of random access memory), and/or circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into larger systems. That is, at least a portion of the devices and/or processes described herein can be integrated into a network processing system via a reasonable amount of experimentation.

The foregoing described aspects depict different components contained within, or connected with different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Embodiments of methods and systems for an ultrasound probe have been described. The following claims are directed to said embodiments, but do not preempt ultrasound probes in the abstract. Those having skill in the art will recognize numerous other approaches to ultrasound probes, precluding any possibility of preemption in the abstract. However, the claimed system improves, in one or more specific ways, the operation of an ultrasound prpobe, and thus distinguishes from other approaches to the same problem/process in how its physical arrangement of a machine system determines the system's operation and ultimate effects on the material environment. The terms used in the appended claims are defined herein in the glossary section, with the proviso that the claim terms may be used in a different manner if so defined by express recitation.

What is claimed is:

1. An ultrasound probe comprising:
a piezoelectric transducer;
a front end analog-to-digital converter;
a first processor;
a transceiver coupled to the first processor which receives and transmits image manipulation signals from user interaction with a plurality of groups of control elements, wherein the plurality of groups of control elements are located on an exterior surface of the ultrasound probe beginning at least 5 mm from a distal end of the ultrasound probe;
wherein interaction with each of the control elements in the plurality of groups of control elements triggers an alert;
and wherein the transceiver transmits image information from the ultrasound probe to a display.

2. The ultrasound probe of claim 1, wherein the alert is at least one of an audible signal, a vibration, a light, or a color change.

3. The ultrasound probe of claim 1, wherein a scan position is maintained during interaction with the control elements.

4. The ultrasound probe of claim 1, wherein a different alert is produced for activation of different control elements.

5. The ultrasound probe of claim 1, wherein the plurality of groups of control elements on the ultrasound probe comprises a slide bar.

6. The ultrasound probe of claim 5, wherein each group in the plurality of groups of control elements on the ultrasound probe further comprises two buttons.

7. The ultrasound probe of claim 6, wherein interaction with the two buttons is a tap or double tap.

8. The ultrasound probe of claim 5, wherein interaction with the slide bar is a swipe, tap, or double tap.

9. The ultrasound probe of claim 8, wherein interaction with the slide bar initiates scrolling through a menu on a user interface.

10. An ultrasound probe which transmits image data wirelessly to a display comprising:
piezoelectric transducer;
a system for analog-to-digital conversion;
a first processor coupled to the piezoelectric transducer;
a transceiver coupled to the first processor which transmits image information to the display;
a battery;
an ultrasound probe housing surrounding the piezoelectric transducer, analog-to digital conversion, first processor, transceiver and battery;
wherein the ultrasound probe housing further comprises a plurality of groups of control elements located about 5 mm from the distal end of an exterior of the ultrasound probe housing; and
wherein interaction with one or more of the control elements in the plurality of groups of control elements sends a signal to the first processor to initiate a stored manipulation function associated with a stored control element function.

11. The ultrasound probe of claim 10, wherein a device function associated with each control element function is static.

12. The ultrasound probe of claim 10, wherein the device function associated with each control element is programmable by a user.

13. The ultrasound probe of claim 10, wherein each of the plurality of groups of control elements in the plurality of groups of control elements performs a same function.

14. The ultrasound probe of claim 10, wherein each of the plurality of groups of control elements in the plurality of groups of control elements performs a different function.

15. The ultrasound probe of claim 10, wherein interaction with a plurality of control elements initiates a function other than the function initiated with a single control element.

16. The ultrasound probe of claim 10, wherein a second group of the plurality of groups of control elements is located on a right side of the exterior of the ultrasound probe housing, wherein the right side is perpendicular to a top side of the exterior of the ultrasound probe housing.

17. The ultrasound probe of claim 10, wherein a second group of the plurality of groups of control elements is located on a left side of the exterior of the ultrasound probe housing, wherein the left side is perpendicular to a top side of the exterior of the ultrasound probe housing.

18. A method of manipulating an ultrasound image comprising:
detecting, from a user, an interaction with at least one control element selected from a plurality of groups of control elements on an exterior surface of an ultrasound probe, wherein placement of each group of control elements is within 5 cm from a distal end of the ultrasound probe;
correlating the interaction with the at least one control element with a stored image manipulation function, wherein each of a plurality of interactions with the at least one control element is correlated to a different image manipulation function;
applying the image manipulation function associated with the interaction with the at least one control element selected from the plurality of groups of control elements to an active ultrasound image; and
wherein subsequent interactions with the at least one control element selected from the plurality of groups of control elements further manipulates the active ultrasound image.

19. The method of claim 18, wherein each group of the control elements in the plurality of groups of control elements performs a same function.

20. The method of claim 18, wherein each group of the control elements in the plurality of groups of control elements performs a different function.
